(19) United States
(12) Patent Application Publication
(10) Pub. No.: US 2008/0319404 A1
(43) Pub. Date: Dec. 25, 2008

(54) Microneedle Devices

(76) Inventors: Mikhail L. Pekurovsky, Bloomington, MN (US); Mitchell A.F. Johnson, Maplewood, MN (US)

Correspondence Address: 3M INNOVATIVE PROPERTIES COMPANY, PO BOX 33427, ST. PAUL, MN, 55133-3427, US

(21) Appl. No.: 12/095,197

(22) PCT Filed: Dec. 19, 2006
(86) PCT No.: PCT/US06/48322

§ 371 Date: May 28, 2008

Related U.S. Application Data
(60) Provisional application No. 60/752,418, filed on Dec. 21, 2005.

(51) Int. Cl. A61M 5/32; A61L 31/16

(52) U.S. Cl. 604/272; 427/2.1

(57) ABSTRACT

Microneedles and microarrays comprising such microneedles are described. The microneedles comprise: a base; a shaft portion extending from the base to a second end distal from the base; a microblade structure extending from the second end; capillary spaces associated with the microblade structure; and a coating in the capillary spaces, the coating comprising an active component. The invention also provides a method for delivering an active component through mammalian skin, comprising: applying the medical device to mammalian skin so that the at least one microneedle is inserted through the stratum corneum and the active component is exposed to interstitial fluids; and allowing the at least one microneedle to remain inserted through the stratum corneum for a sufficient amount of time to dissolve the active component. In another aspect, the invention provides a method for applying an active component to a microneedle, comprising: providing a microneedle, comprising: a base, a shaft portion extending from the base to a second end distal from the base, a microblade structure extending from the second end, and capillary spaces associated with the microblade structure; applying a liquid coating to at least the microblade structure of the microneedle, the liquid coating comprising an active component; and drying the liquid coating to provide a dried coating of active component, the dried coating located at least within the capillary spaces of the microblade structure.

### CROSS-REFERENCE TO RELATED APPLICATIONS

This application claims priority to U.S. Provisional Application Ser. No. 60/752,418, filed Dec. 21, 2005, which is incorporated herein by reference in its entirety.

### FIELD

The present invention relates to microneedles, microarrays that comprise microneedles and to methods for the manufacture and the use of such microneedles.

### BACKGROUND

Pharmaceutical compositions, vaccines, drugs, therapeutic substances, etc. (“active components”) may be delivered into the body through the skin in any of a number of different ways. The main barrier to the transport of active components through the skin is the outermost layer of the skin known as the stratum corneum. Consequently, if an active component is to be delivered through the skin, the active component must be provided with a pathway through the stratum corneum. Active components, can be delivered through the skin by injection using a hypodermic syringe with a hollow needle that will puncture the stratum corneum and deliver the active component beneath the skin. Other means for the delivery of certain active components include transdermal patches, ointments or lotions as well as microneedle arrays.

Microneedle arrays provide a means for the delivery of active components through the skin. Microneedle arrays, or microarrays, include a plurality of small piercing elements or microneedles capable of piercing the stratum corneum upon contact, thereby creating a plurality of microscopic slits that serve as passageways through which active components can be delivered into the body. Active component may be associated with the microarray in as a dry or dried solid, a liquid, or the like. In some arrangements, the microarray may be coated with a liquid containing an active component that is thereafter dried to provide the active component as a coating on the microarray that can be delivered directly through the skin after the stratum corneum has been punctured. Microarrays can be provided as transdermal patches in a construction that permits adhesive attachment of the microarray to the skin of a mammal.

### SUMMARY

Improvements are provided in the construction of microneedles and in microarrays comprising such microneedles. The improvements include microneedles having microblade structures thereon. The microblade structures include features that can accept and retain a coating of one or more active components.

In an aspect, the present invention provides a microneedle, comprising: a base; a shaft portion extending from the base to a second end distal from the base; a microblade structure extending from the second end; capillary spaces associated with the microblade structure; and a coating in the capillary spaces, the coating comprising an active component.

In another aspect, the invention provides a medical device, comprising: an array retaining member comprising a support surface; at least one microneedle as previously described extending from the support surface.

In still another aspect, the invention provides a method for delivering an active component through mammalian skin, comprising: Applying the medical device as described above to mammalian skin so that the at least one microneedle is inserted through the stratum corneum and the active component is exposed to interstitial fluids; and allowing the at least one microneedle to remain inserted through the stratum corneum for a sufficient amount of time to dissolve the active component.

In still another aspect, the present invention provides a method for applying an active component to a microneedle, comprising:
(A) Providing a microneedle, comprising:
   a base,
   a shaft portion extending from the base to a second end distal from the base,
   a microblade structure extending from the second end, and capillary spaces associated with the microblade structure;
(B) Applying a liquid coating to at least the microblade structure of the microneedle, the liquid coating comprising an active component; and
(C) Drying the liquid coating to provide a dried coating of active component, the dried coating located at least within the capillary spaces of the microblade structure.

In the description of embodiments of the invention, certain terminology is used which shall be understood to have the meanings set forth herein.

“Aspect ratio,” in reference to the microneedles described herein, refers to the ratio of the height of the microneedle (above the surface surrounding the base of the microneedle) to the maximum or longest straight-line dimension of the base of the microneedle, (measured at the surface occupied by the base of the microneedle).

“Capillarity” or “capillary action” means the action by a liquid when in contact with a solid wherein the liquid is elevated or depressed depending on the relative attraction of the molecules of the liquid for each other and for those of the solid.

“Capillary spaces” refer to specific areas bounded by at least two surfaces of a solid where capillary action occurs.

“Microblade structure” refers to the structure of a portion of a microneedle providing one or more microscopic cutting surfaces capable of piercing mammalian skin.

“Active component” refers to one or more pharmacologically effective agents such as non-immunogenic drugs, immunogenic substances such as vaccines, salts thereof, and the like. Active components described herein may or may not be present at pharmaceutically or clinically effective amounts.

Those skilled in the art will better understand the features of the embodiments of the invention upon further consideration of the remainder of the disclosure, including the various Figures taken together with the detailed description and the appended claims.

### BRIEF DESCRIPTION OF THE DRAWINGS

In describing embodiments of the invention herein, reference is made to the various Figures in which like reference numerals indicate like structures and wherein:

FIG. 1 is a perspective view of one embodiment of a microarray patch device according to the present invention;

FIG. 2 is a perspective view of a microneedle having a microblade structure according to an embodiment of the present invention;

FIG. 3 is a perspective view of a microneedle having a microblade structure according to another embodiment of the present invention;

FIG. 4 is a perspective view of a microneedle having a microblade structure according to another embodiment of the present invention; and

FIG. 5 is a perspective view of a microneedle having a microblade structure according to still another embodiment of the present invention.

### DETAILED DESCRIPTION

The invention provides microneedles for a microneedle array suitable for the delivery of active components into or through mammalian skin. In the embodiments described herein, the microneedles of the invention include capillary spaces at or near the tip of the microneedle. In some embodiments, the invention provides microneedles with capillary spaces that are coated with a dried composition comprising an active component. In some embodiments, the capillary spaces are provided in conjunction with cutting blades capable of cutting into the stratum corneum. In some embodiments, the invention provides microarrays that comprise the microneedles of the invention.

In embodiments of the invention, microneedles are provided that comprise a base portion proximal to a supporting substrate and a tip section distal from the supporting substrate. The tip portion includes a microblade structure that is different than the structure of the rest of the microneedle. The microblade structure on the tip of the microneedle includes capillary surfaces and provides at least one microscopic cutting edge capable of piercing the stratum corneum.

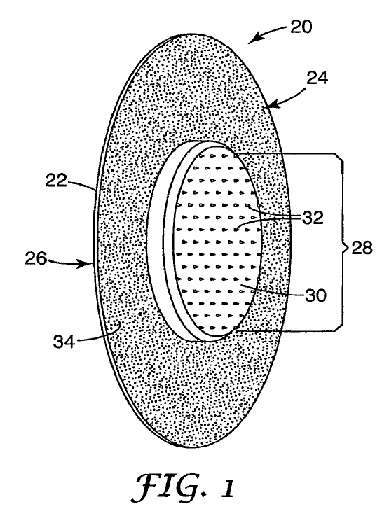

Referring to the various Figures, embodiments of the invention are shown and will now be described. Referring to FIG. 1, a patch 20 is depicted. The patch 20 includes an extension member 22 with a first major surface 24 and a second major surface 26. The first major surface 24 of the extension member 22 comprises a first portion having an array retaining member 28 extending therefrom. The array retaining member 28 includes an array surface 30 having a plurality of microneedles 32 extending from the surface 30. A second portion of the first major surface 24 of extension member 22 includes a layer of pressure sensitive adhesive 34 disposed thereon. The pressure sensitive adhesive is provided on the second portion of the surface 24 of the extension member 22 to facilitate the adhesive attachment of the patch 20 to mammalian skin when the microneedles 32 are inserted through the stratum corneum. In some embodiments, the adhesive layer 34 will extend from the first major surface 24 of the extension member 22 to a height less than the height of the array surface 30. While the patch 20 is depicted in a circular configuration, it will be appreciated that the patch 20 may be configured in any useful or ornamental configuration desired.

Microneedles according to the present invention may be characterized, in part, by their aspect ratio. In embodiments of the invention, the microneedles can have an aspect ratio as low as 2:1. In other embodiments, the aspect ratio is greater than 2:1, and in some embodiments the microneedles can have an aspect ratio of 3:1 or higher.

In embodiments, the microneedles are provided as a single array comprising a multitude of individual microneedles which are manufactured integrally with the device of the invention. In some embodiments, the microneedles can initially be provided separately and later added to the substrate during the manufacture or assembly of the device.

The microneedles may be manufactured from any of a variety of materials, and the actual material selected for a particular microneedle array can be based on a variety of factors including the ability of the material to accurately reproduce the desired microneedle pattern; the strength and toughness of a particular material when formed into the microneedles; the compatibility of a material with mammalian skin; the compatibility of a material with body fluids expected to contact the microneedle array, etc.

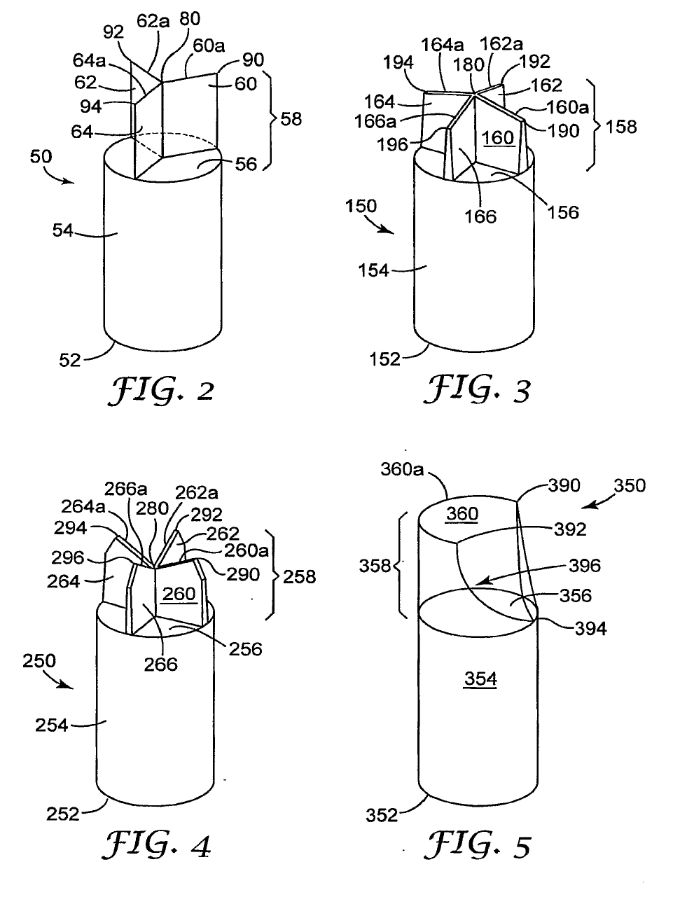

Referring to FIG. 2, an embodiment of a microneedle 50 according to the invention is shown and will now be described. The microneedle 50 may be part of a microneedle array comprising identically constructed microneedles or microneedles that comprise different constructions than the microneedle 50. As shown, microneedle 50 includes a first end or base 52 affixed to and extending from a supporting substrate (not shown). Shaft 54 extends from the first end or base 52 (and supporting substrate) in a substantially orthogonal manner, forming a second end 56. Microblade structure 58 is provided on the second end 56 so that the microblade structure 58 essentially forms the tip of the microneedle 50. Microblade structure 58 includes a plurality of wing members 60, 62 and 64 extending from a common central axis that terminates at center point 80. Each wing member 60, 62 and 64 include, respectively, an upper edge 60a, 62a and 64a. The edges 60a, 62a and 64a are microscopic cutting edges capable of slicing through the stratum corneum.

In embodiments of the invention, microblade structure 58 is configured so that all three wing members 60, 62 and 64 are identical and with edges 60a, 62a and 64a being coplanar and extending from a common first end corresponding to the center point 80. Each edge terminates at a second end with edge 60a terminating at second end 90, edge 62a terminating at second end 92 and third edge 64a terminating at second end 94. In some embodiments, the microblade structure 58 is configured so that the common first end (center point 80) of wings 60, 62 and 64 is the distal-most point from the substrate supporting the base 52. In such a construction, the center point 80 would be first to initiate contact with and subsequently penetrate the stratum corneum when the microneedle 50 is pressed against the surface of the skin. In some embodiments, the second ends 90, 92, 94 of edges 60a, 62a and 64a, respectively, are farther away from the base 50 than the center point 80. In such a construction, the second ends 90, 92 and 94 would be first (e.g., prior to the center point 80) to simultaneously initiate contact with the skin to pierce the stratum corneum. In other embodiments, any one or two of the second ends 90, 92 and 94 may form the distal-most point of the microneedle 50.

Microblade structure 58 is arranged so that the penetration of the wings 60, 62 and 64 into the skin forms v-shaped cut sections in the skin between the individual cuts formed by each of the wings 60, 62 and 64. In this manner, insertion of the microneedle 50 into the stratum corneum requires that only a small area of the skin has to be stretched so that resistance to penetration is minimized. Although the three wings 60, 62 and 64 are depicted as identically configured, it will be appreciated that they need not be identical. Moreover, embodiments are contemplated that comprise only one wing-like member or that comprise two wing-like members. Additionally, angles between wing members, while depicted as essentially equal, may be different in some embodiments of the invention. All such embodiments and variations thereof are considered to be within the scope of the present invention.

In another aspect, the v-shaped spaces between the wings 60 and 64, wings 60 and 62 and wings 62 and 64 comprise capillary spaces. In embodiments of the invention, the capillary spaces include a coating of active component therein. In this regard, the materials used to form the microneedle 50 and especially the microblade structure 58 are compatible with a liquid composition used for coating such an active component onto the microblade structure 58. In embodiments where the active component is initially provided in a coatable aqueous composition, the microblade structure 58 comprises a hydrophilic material. In some embodiments, the shaft 54 and the wings 60, 62 and 64 are made of the same material(s). In other embodiments, the shaft 54 and the wings 60, 62 and 64 comprise different materials. When applied in a liquid coating, the composition tends to remain within the capillary spaces due to the relative attraction of the molecules of the liquid composition for each other and for those of the solid materials used to form the wings 60, 62 and 64. The solvent of the liquid composition (e.g., water) is thereafter removed by drying or is allowed to evaporate to leave a dried coating of active component within the capillary spaces on the microblade structure 58.

Materials suitable for use in the construction of the microblade structures of the present invention include those selected from materials such as acrylonitrile-butadiene-styrene (ABS) polymers, polyphenyl sulfides, polycarbonates, polypropylenes, acetals, acrylics, polyetherimides, polybutylene terephthalates, polyethylene terephthalates as well as other known materials and combinations of two or more of the foregoing. A suitable method for molding the microarrays of the invention is described in copending PCT publication WO05/082596, filed Feb. 22, 2005.

Referring to FIG. 3, another embodiment of a microneedle 150 according to the invention is shown. The microneedle 150 may be part of a microneedle array comprising identically constructed microneedles or microneedles that comprise constructions different than the construction of microneedle 150. As shown, microneedle 150 includes a base 152 affixed to and extending from a supporting substrate (not shown). The shaft 154 extends from the base 152 (and supporting substrate) in a substantially orthogonal manner, forming a second end 156. Microblade structure 158 is provided on the second end 156 and the microblade structure 158 essentially forms the tip of the microneedle 150.

Microblade structure 158 includes four identical wings 160, 162, 164 and 166 with corresponding edges 160a, 162a, 164a and 166a that serve as cutting edges and are capable of slicing through the stratum corneum when the microneedle 150 is pressed against the skin. In the depicted embodiment, the microblade structure 158 is configured so that the first end of each wing 160, 162, 164 and 166 is center point 180. In the depicted configuration, the center-point 180 is also is the distal-most point from the substrate supporting the base 152. Consequently, when the microneedle is pressed against the skin, the center point 180 would be first to initiate contact with and subsequently penetrate the stratum corneum when while the second end points 190, 192, 194 and 196 of edges 160a, 162a, 164a and 166a would contact the skin after the center point 180. In another aspect, the v-shaped spaces between the wings 160 and 162, wings 162 and 164, wings 164 and 166, and wings 166 and 160 are capillary spaces, as previously described. In other aspects of the present embodiment, the capillary spaces include a coating of active component therein. Although the angles between the four wing members are depicted as being of essentially equal measure, these angles and the spaces defined therewithin may be different from one another in some embodiments of the invention. All such embodiments and variations thereof are considered to be within the scope of the present invention.

Referring to FIG. 4, another embodiment of a microneedle 250 is shown. The microneedle 250 may be part of a microneedle array comprising identically constructed microneedles or microneedles that comprise different constructions than the microneedle 250. As shown, microneedle 250 includes a base 252 affixed to and extending from a supporting substrate (not shown). The shaft 254 extends from the base 252 (and supporting substrate) in a substantially orthogonal manner, forming a second end 256. Microblade structure 258 is provided on the second end 256 and the microblade structure 258 essentially forms the tip of the microneedle 250.

Microblade structure 258 includes wings 260, 262, 264 and 266 with corresponding edges 260a, 262a, 264a and 266a which serve as cutting edges that are capable of slicing through the stratum corneum. In the depicted embodiment, the microblade structure 258 is configured so that the tips 290, 292, 294 and 296 of the wings 260, 262, 264 and 266 are the distal-most points from the substrate supporting the base 252. In such a construction, the tips 290, 292, 294 and 296 would be first to initiate contact with and thereafter penetrate the stratum corneum when the microneedle 250 is pressed against the surface of the skin. In another aspect, the v-shaped spaces between the wings 260 and 262, wings 262 and 264, wings 264 and 266, and wings 266 and 260 are capillary spaces, as previously described. In other aspects of the present embodiment, the capillary spaces include a coating of active component therein.

In other aspects of the embodiment, microblade structure 258 may be configured so that all four edges 260a, 262a, 264a and 266a are essentially coplanar. While the four wing members are depicted in FIG. 4 as essentially identical, some embodiments of the invention may comprise four non-identically configured wing members. Moreover, the angles between wing members, while depicted as essentially equal, may be different in some embodiments of the invention. In some embodiments, the microblade structures may comprise more than four wing members which may or may not be identical to one another, may or may not be spaced from one another in a uniform manner with all of the capillary spaces holding essentially the same volume of liquid or dried coating therein. Regardless of such possible variations in construction, all such embodiments and variations thereof are considered to be within the scope of the present invention.

Referring to FIG. 5, another embodiment of a microneedle 350 is shown. The microneedle 350 may be part of a microneedle array comprising identically constructed microneedles or microneedles that comprise different constructions than the microneedle 350. As shown, microneedle 350 includes a base 352 affixed to and extending from a supporting substrate (not shown). The shaft 354 extends from the base 352 (and supporting substrate) in a substantially orthogonal manner, forming a second end 356. Microblade structure 358 is provided on the second end 356 and the microblade structure 358 essentially forms the tip of the microneedle 350.

Microblade structure 358 is configured as an opened cylinder having a curved partial wall 360 terminating in a distal cutting edge 360a. Cutting edge 360a is bounded by first end 390 and second end 392. On the side of the microblade structure 358 opposite that of the cutting edge 360a, a v-shaped opening is provided between the tip 390 and 392 of the edge 360a, the opening bounded by edges extending from each tip 390, 392 to midpoint 394 at or near the second end 356 of the shaft 354. In the depicted embodiment, the microblade structure 358 is configured so that the tips 390, 392 and the edge 360a are all equidistant from the substrate that supports base 352. In such a construction, the entire edge 360a initiates contact with the stratum corneum when the microneedle 350 is pressed against the skin. In other aspects of the present embodiment, the tips 390, 392 can be configured to be the distal most points on the microneedle 350 so that the tips 390, 392 would be first to initiate contact with the stratum corneum when the microneedle 350 is pressed against the surface of the skin. In another aspect, the space 396 within the inner curved portion of the microblade 358 is a capillary space, as previously described. In other aspects of the present embodiment, the capillary space 396 include a coating of active component therein.

Other configurations for the microneedles of the invention are contemplated. In general, the invention includes microneedles and microneedle arrays that that comprise microneedles having a microblade structure with capillary spaces associated with the microblade structure. The microblade structures of the invention comprise a coating of active component associated with the capillary spaces on the microblade structure. The active component may be applied to the microblade structures of the invention in a manner resulting in the capture (e.g., by capillary action) of the active component in the capillary spaces of the microblade structure. Typically, a liquid composition is prepared that comprises one or more active components in a volatile solvent such as water, lower alcohols or the like. The composition is applied to the microneedle in a manner that exposes the microblade to the composition so that the composition is deposited on the microblade structure. In some embodiments, the liquid composition is “pinned” on the microblade structure so that the entire microblade structure, including the capillary spaces, is encapsulated in the composition. In the foregoing embodiment, the capacity of the capillary spaces is exceeded, and the resulting coating of active component will extend outside of the capillary spaces and cover all or nearly all of the microblade structure. In other embodiments, the composition is not pinned on the microblade structure, but the microblade structure is exposed to the composition in a manner that facilitates filling or partial filling of the capillary spaces. In this embodiment, the composition is retained within the capillary spaces of the microblade.

Following exposure to the composition, the microblade structure is dried. Drying may be accomplished by exposure to elevated temperatures (e.g., oven heat) in order to drive off the volatile components of the composition such as solvent(s) and the like. Drying may also be accomplished at ambient conditions (e.g., 20° C.-25° C.) wherein the microblade structure is not subjected to elevated temperatures but solvents and other volatile components are allowed to evaporate from the microblade. Regardless of the method used to dry the microneedles, the resulting microneedle includes a microblade structure having a dried coating of active component. In some embodiments, the entire microblade structure may be coated with active component. In other embodiments, the coating of active component is principally within the capillary spaces. In a microarray, the thus treated microneedle may be applied to the skin to pierce the stratum corneum and thereby deliver the active component to a mammalian patient.

In some embodiments of the invention, the microneedles of the invention may be coated according to the method described in copending PCT publication WO06/055844, claiming priority to U.S. provisional patent application No. 60/629,187, filed on Nov. 18, 2004, the entire disclosure of which is incorporated herein by reference thereto. In other embodiments, the microneedles of the invention may be coated according to the method described in copending PCT publication WO06/055799, claiming priority to U.S. provisional patent application No. 60/629,209, the entire disclosure of which is incorporated herein by reference thereto.

In the delivery of an active component through mammalian skin, a microarray, as previously described, is provided comprised of one or more microneedles, each microneedle having a microblade structure thereon and a coating of active component in the capillary spaces of the microblade structure. The microarray is applied to the surface of mammalian skin in a manner that facilitates the insertion of the microneedles through the stratum corneum to expose the microblade structures and the coating of active component to interstitial fluids. The microarray may be left with the microneedles inserted through the stratum corneum for a sufficient length of time to adequately dissolve the active component(s) in interstitial fluids. In this manner, the active component is delivered transdermally.

While embodiments of the invention have been described, it will be appreciated that insubstantial modifications, not presently foreseeable by those of reasonable skill in the art, may be made which represent equivalents to the embodiments described and claimed herein.

What is claimed:

1. A microneedle, comprising:
a base;
a shaft portion extending from the base to a second end distal from the base;
a microblade structure extending from the second end;
capillary spaces associated with the microblade structure; and
a coating in the capillary spaces, the coating comprising an active component.

2. The microneedle according to claim 1 wherein the microblade structure comprises at least one microscopic cutting surface capable of piercing mammalian stratum corneum.

3. The microneedle according to claim 1 wherein the microblade structure comprises a plurality of wing members extending outwardly from a common central axis, each of the plurality of wing members has a corresponding upper edge capable of slicing through the stratum corneum, the upper edges extending from a common first end at the central axis but terminating at different second ends.

4. The microneedle according to claim 3 wherein the microblade structure comprises a first wing member having a first upper edge, a second wing member having a second upper edge and a third wing member having a third upper edge.

5. The microneedle according to claim 4 wherein the first, second and third upper edges extend from the common first end and the first, second and third upper edges are coplanar.

6. The microneedle according to claim 4 wherein the common first end is a point, the common first end being the distal-most point from the base.

7. The microneedle according to claim 4 wherein the common first end is a point, the different second ends being coplanar and comprising the distal-most points from the base.

8. The microneedle according to claim 4 further comprising a fourth wing member having a fourth upper edge.

9. The microneedle according to claim 8 wherein the first, second, third and fourth upper edges extend from the common first end and the first, second, third and fourth upper edges are coplanar.

10. The microneedle according to claim 8 wherein the common first end is a point that is the distal-most point from the base.

11. The microneedle according to claim 8 wherein the common first end is a point, the different second ends being coplanar and comprising the distal-most points from the base.

12. The microneedle according to claim 1 wherein the coating is substantially within the capillary spaces.

13. The microneedle according to claim 1 wherein the coating extends outside of the capillary spaces.

14. The microneedle according to claim 13 wherein the coating covers the entire microblade structure.

15. A method for applying an active component to a microneedle, comprising:
(A) providing a microneedle, comprising:
a base,
a shaft portion extending from the base to a second end distal from the base,
a microblade structure extending from the second end, and
capillary spaces associated with the microblade structure;
(B) applying a liquid coating to at least the microblade structure of the microneedle, the liquid coating comprising an active component; and
(C) drying the liquid coating to provide a dried coating of active component, the dried coating located at least within the capillary spaces of the microblade structure.

16. The method of claim 15 wherein the microblade structure comprises at least one microscopic cutting surface capable of piercing mammalian skin.

17. The method of claim 16 wherein the microblade structure comprises a plurality of wing members extending outwardly from a common central axis, each of the plurality of wing members has a corresponding upper edge capable of slicing through the stratum corneum, the upper edges extending from a common first end at the central axis but terminating at different second ends.

18. The method of claim 17 wherein the microblade structure comprises a first wing member having a first upper edge, a second wing member having a second upper edge and a third wing member having a third upper edge.

19. The method of claim 17 wherein the first, second and third upper edges extend from the common first end and the first, second and third upper edges are coplanar.

20. The method of claim 18 wherein the common first end is a point, the common first end being the distal-most point from the base.
